(19) United States
(12) Patent Application Publication
(10) Pub. No.: US 2007/0299517 A1
(43) Pub. Date: Dec. 27, 2007

(54) Articular cartilage implant

(75) Inventors: Twana Davisson, Montclair, NJ (US); Marc Long, Denville, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/471,955

(22) Filed: Jun. 21, 2006

(51) Int. Cl. A61F 2/02

(52) U.S. Cl. 623/11.11; 623/17.11; 623/18.11; 623/23.61

(57) ABSTRACT

The invention is directed to the repair of articular cartilage in joints and includes an articular cartilage graft, a method of producing the articular cartilage graft, and a method of repairing an articular cartilage defect using the articular cartilage graft. In its preferred embodiment, the articular cartilage graft of the present invention comprises a collagen portion with structure or architecture similar to, or substantially the same as, native human articular cartilage.

### FIELD OF THE INVENTION

The present invention relates to the field of medical technology and is generally directed to the treatment of cartilage or cartilage and bone defects through the use of grafts.

### BACKGROUND OF THE INVENTION

The surfaces of normal human and animal joints are covered by articular cartilage. The function of articular cartilage in a joint is to provide a durable low friction surface that distributes mechanical forces and protects the joint's underlying bone. Loss of or damage to articular cartilage usually leads to both painful arthritis and decreased motion in the damaged joint. Damage to articular cartilage and joint surfaces can be caused by traumatic injury or disease.

Articular cartilage primarily consists of a collagen matrix, proteoglycans, and water with a small number of chondrocytes distributed throughout the matrix. Chondrocytes are the cells responsible for the production of articular cartilage.

The structure of the collagen matrix in articular cartilage is unique. It forms a pattern of overlapping leaves that are arranged vertically close to the bone and then curve to become parallel to the joint surface as the collagen moves away from the bone. Jeffery, A. K. et al., September: 73(5) J Bone Joint Surg. Br. 795-801 (1991). As a result, articular cartilage in joints has a specific orientation and direction depending on its location. This specific orientation can be visualized by looking at the split-line pattern produced when cartilage is penetrated with a sharp object coated in ink. Leo, B. M. et al., July: 20 Suppl. 2 Arthroscopy, 39-45 (2004). Some studies have suggested that there is a relationship between the orientation of collagen fibers and the strength of articular cartilage. Leo et al. (2004).

Different types of collagen can be found in varying amounts in the collagen matrix, depending on the type of tissue. For example, hyaline cartilage, which is found predominantly in articulating joints, is composed mostly of type II collagen with small amounts of types V, VI, IX, X, and XI collagen also present. On the other hand, fibrocartilage, which can also be found in joints, is primarily composed of type I collagen. Additionally, the fibrocartilaginous tissue that sometimes replaces damaged articular cartilage is composed of type I collagen.

A layer of calcified cartilage separates hyaline cartilage from bone in a joint. In addition, the calcified cartilage layer attaches the hyaline cartilage to the bone. The boundary line between the calcified cartilage and hyaline cartilage is called the tidemark.

Since cartilage is an avascular tissue, meaning that it lacks nerves and blood vessels, articular cartilage has very limited regenerative capabilities compared to other tissues. Consequently, the healing of damaged joint cartilage results in a fibrocartilaginous repair tissue that lacks the structure and biomechanical properties of normal cartilage. Over time, the repair tissue degrades and leaves damaged joint cartilage, which causes osteoarthritis and reduced movement in the joint.

Damaged articular cartilage creates a number of medical problems for the general population, particularly adults. For example, osteoarthritis results in the disability and impairment of many middle-aged and older individuals. There are also significant economic, social, and psychological costs. Individuals with cartilage defects and osteoarthritis often experience debilitating pain and a reduced range of joint movement that makes normal daily activities extremely difficult.

Although a number of different therapeutic methods are currently being used to treat articular cartilage defects, they have only been marginally successful. Some of the current treatments include lavage, arthroscopic debridement, and repair stimulation. However, these therapeutic methods either provide only temporary pain relief or have shown limited clinical efficacy.

Other treatment methods involve grafting the defect site with artificial materials, autografts, allografts, or xenografts. Examples of different grafts and grafting methods can be found in U.S. Pat. Nos. 5,944,755; 5,782,915; 6,858,042; 2003/0229400; and 2004/0230303. One particular grafting method, called mosaicplasty, has shown some clinical efficacy. Mosaicplasty involves removing small autologous osteochondral plugs from low weight bearing sites in a patient's joint. The osteochondral plugs are then grafted into a mosaic of holes drilled into the patient's articular cartilage defect site. Some patients who have undergone mosaicplasty have reported decreased pain and improved joint function. Marcacci, M. et al., Arthroscopy 21(4): 462-470 (2005).

Although all of the above methods have had some clinical success, each one of these therapeutic methods suffer from one or more of the following disadvantages: the risk of patient immune response or disease transmission; limited availability of osteochondral autograft sites; lack of implant adhesion to the defect site; implant deterioration; lack of long-term efficacy; donor site morbidity; patient discomfort; and the failure to restore normal joint function.

There continues to be a need for an osteochondral implant that restores normal joint function and addresses the limitations of the current therapies.

### SUMMARY OF THE INVENTION

The present invention is directed towards a graft that can be used to repair articular cartilage. The invention also includes a method of producing the graft and a method of treating cartilage defects using the graft. Accordingly, the present invention provides a graft that has substantially the same three-dimensional collagen structure or architecture as native human articular cartilage.

The present invention also provides a method of making an articular cartilage graft that includes removing at least a portion of a joint from a human cadaver, where the removed joint portion comprises cartilage and attached bone. The removed joint portion, or graft, is then treated (for example, with a cross-linker or by glycation) to stabilize the graft's collagen scaffold structure. Consequently, the graft of the present invention maintains substantially the same three dimensional collagen scaffold structure as the articular cartilage in the native human cadaver joint. The articular cartilage grafts provided by any of the described methods are also included in the present invention.

The present invention also comprises a method for making an articular cartilage graft that includes removing at least a portion of a joint from an animal, where the removed joint portion comprises cartilage and attached bone. The removed joint portion, or graft, is then treated (for example, with a cross-linker or by glycation) to stabilize the graft's collagen scaffold structure. At least a portion of the proteoglycans are also removed from the graft, such that at least one of the mechanical properties of the cartilage portion of the graft is substantially different than the mechanical properties found in the native articular cartilage. The articular cartilage graft provided by above described method is also included in the present invention.

In various embodiments of the invention, the removed joint portion may comprise a focal core, a full or partial intact condyle, a full or partial intact patellar groove, a portion of a joint, or an entire joint. The removed joint portion may be taken from any joint, including, but not limited to, the knee, elbow, ankle, hip, shoulder, wrist, or spine. The bone portion of the graft may also be removed to produce a scaffold that is only cartilage. Alternatively, a layer of articular cartilage devoid of bone may be used to generate the graft. The graft may provide chondral, osteochondral, partial, or full repair of joint cartilage.

Some embodiments of the invention comprise devitalizing the graft, for example, by freezing the graft; treating the graft with alcohol; subjecting the graft to gamma radiation; or subjecting the graft to freeze-thaw cycles. Other embodiments comprise demineralizing the bone portion of the graft, for example, by treating the graft with guanidine hydrochloride, hydrochloric acid, or basic EDTA.

Some embodiments of the inventive method also include removing at least a portion of the cellular debris, for example, by treating the graft with choloroform.

Other embodiments include removing at least a portion of the proteoglycans and non-collagenous materials from the graft, for example, by processing with enzymes.

In some embodiments, the method of the present invention comprises seeding the graft with cells. Cells that may be used to seed the graft include, but are not limited to, stem cells, mesenchymal cells, bone marrow cells, synovial cells, progenitor cells, osteoblasts, fibroblasts, chondroblasts, and chondrocytes.

Further embodiments include adding biological agents to the graft. For example, growth factors, chondroinductive agents, pain-killers, proteins, non-steroidal anti-inflammatory drugs, or antibiotics may all be added to the graft either separately or in combination with one another.

The invention also comprises a method of repairing cartilage defects using the grafts by joining the graft to or inserting the graft into either a partial or full-thickness cartilage defect in a joint. In various embodiments, the graft may be implanted either arthroscopically or through an open incision.

In some embodiments, a surgeon can match the collagen orientation of the graft to the split-line orientation of the cartilage defect. Matching the collagen orientation of the graft and defect is facilitated by choosing grafts from similar anatomical sites as the defect, but this is not required. Another embodiment of the invention comprises matching the curvature of the graft's outer surface with the curvature of the cartilage defect. Instrumentation or imaging techniques to measure and match the curvature may also, optionally, be packaged with the graft material as part of the procedure.

In further embodiments, a surgeon may press-fit the graft into the defect or use an anchor or adhesive to affix the graft to the defect site. In certain embodiments, different anchors or adhesives may also be used, separately or in combination with one another, to affix the graft to the defect site. The anchors may also, optionally, be an integral part of the graft or separate from the graft.

### BRIEF DESCRIPTION OF THE DRAWINGS

FIG. 1 shows the collagen orientation in devitalized osteochondral goat tissue, based on polarized light microscropy. The collagen orientation in these grafts is similar to that of native human articular cartilage.

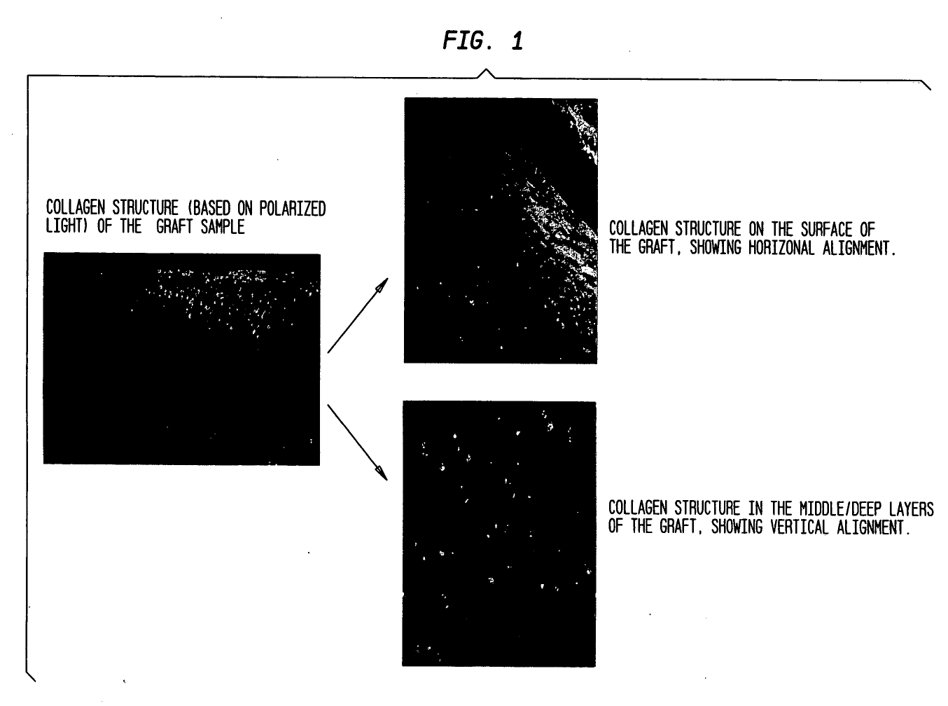

FIG. 2 shows the proteoglycan content of grafts generated using caprine tissue. Samples A and B were digested in a hyaluronidase/trypsin solution Samples C and D were digested with hyaluronidase alone.

### DETAILED DESCRIPTION

The present invention is directed to the repair of articular cartilage in joints and includes an articular cartilage graft, a method of producing the articular cartilage graft, and a method of repairing an articular cartilage defect using the articular cartilage graft. In its preferred embodiment, the articular cartilage graft of the present invention comprises a collagen portion with structure or architecture similar to, or substantially the same as, native human articular cartilage.

The invention also provides a method for producing an articular cartilage graft from animals. The process of making a graft according to the present invention comprises removing at least a portion of a joint from an animal, where the joint portion, or graft, comprises bone and cartilage; treating the graft to stabilize the native structure of the cartilage; and removing at least a portion of the proteoglycans in the cartilage portion of the graft, such that one or more mechanical properties of the cartilage is altered. Preferably, the bone and cartilage portion of the joint are attached. In its preferred embodiment, the articular cartilage graft of the present invention comprises a collagen portion with structure or architecture similar to, or substantially the same as, native articular cartilage.

The structure of the collagen matrix in articular cartilage is unique. It forms a pattern of overlapping leaves that are arranged vertically close to the bone and then curve to become parallel to the joint surface as the collagen moves away from the bone. The collagen fibers are arranged vertically in the intermediate zone and curve near the superficial zone to become parallel to the joint surface. As a result, articular cartilage in joints has a specific orientation and direction depending on its location. This specific orientation can be visualized by looking at the split-line pattern produced when cartilage is penetrated with a sharp object coated in ink.

The terms “structure” and “architecture” of the collagen in the graft refers to the three dimensional organization of collagen that is present in articular cartilage, particularly human articular cartilage.

By “similar” and “substantially the same as” the architecture or structure of native human articular cartilage, it is meant that the orientation of collagen in the graft does not have to be identical to native human articular cartilage. However, the collagen in the graft should have at least one of the following arrangements: a curved-leaf structure similar to that found in native human articular cartilage; a substantially vertical arrangement in the intermediate zone with curving near the superficial zone; and a similar split-line orientation as the native human articular cartilage. In some embodiments, the graft contains collagen structure which is similar to all three arrangements. In other embodiments, the graft contains collagen structure which is similar to only two of these arrangements. In other embodiments, the graft contains collagen structure which is similar to only one of these arrangements.

The stabilized three-dimensional collagen structure of the graft may be composed of type I collagen, type II collagen, type III collagen, type V collagen, type VI collagen, type IX collagen, type X collagen, type XI collagen, or any other collagen type. The collagen structure of the graft may also be composed of any mixture of collagen types and any specific collagen type may be present or absent from the graft. The amounts of specific collagen types present in the graft may vary. For example, the graft may be composed of about 5 to about 100%, about 10 to about 90%, about 20 to about 80%, about 30 to about 70%, or about 40 to about 60% type II collagen. Likewise, in some embodiments, the graft may also be composed of about 5 to about 100%, about 10 to about 90%, about 20 to about 80%, about 30 to about 70%, or about 40 to about 60% type I collagen.

The invention described herein is advantageous over the prior art because it provides an articular cartilage graft that can potentially restore normal joint function without an immune response. Preserving the three dimensional collagen structure is expected to promote and accelerate cartilage growth within the graft, thereby improving the long-term quality of the cartilage tissue in the surgical site. It will also allow a surgeon to match the orientation of the collagen in the graft with the split line orientation of the cartilage defect site. Matching the collagen orientation of the graft with the collagen orientation observed at the defect site is expected to increase the quality of repair at the implant site.

The graft of the present invention can be used as both a scaffold for ex vivo cartilage growth or as an implant used to repair articular cartilage in a joint that is implanted alone or in combination with cells and/or biological factors at the time of surgery. The graft may be used for chondral, osteochondral, partial or full repair of joint defects.

The graft may include both cartilage and bone, or only cartilage.

The invention also provides a method for producing an articular cartilage graft. The process of making a graft according to the present invention comprises removing a portion of a joint from a human cadaver, where the joint portion, or graft, comprises bone and cartilage; and treating the graft to stabilize the native structure of the cartilage. Preferably, the bone and cartilage portion of the joint are attached.

The bone portion helps maintain the three dimensional architecture of the collagen portion prior to the treatment for collagen stabilization, and can promote maintenance in the cartilage defect site upon implantation. The cartilage portion either partially or completely repairs the cartilage defect upon implantation.

In other embodiments, the joint portion does not contain any bone.

The joint portion may be removed from any type of human cadaver, without regard to variations among human cadaver types. For example, the joint portion can be removed from a male or female cadaver, having any age, height, or weight, or any other specific characteristic found in humans.

Likewise, the joint portion may be removed from any animal, without regard to variation as to animal types. The animal may be any vertebrate. Preferably, the animal is a mammal. Suitable animals include pigs, cows, horses, dogs, cats, sheep, goats, rodents (esp. mice or rats), emus and ostriches.

Any method may be used to remove the joint portion from the cadaver or animal, provided the removal method does not significantly disrupt the three dimensional structure of the collagen of the joint portion. Preferably, the joint portion is removed under sterile conditions.

After removal, the graft may be washed in a suitable buffer. Suitable buffers include water (preferably sterile), phosphate buffered saline, physiologic saline, and diluted alcohol solution.

The size of the bone portion and/or the cartilage portion can vary depending on the size of the cartilage defect. For example, the bone portion can be anywhere from about 1 mm to the size of an entire joint depending on the size of the defect. Likewise, the size of the cartilage portion can be anywhere from about 1 mm to the size of the entire joint, depending on the size of the defect to be repaired. The bone portion and cartilage portion of the removed joint do not have to be of equal size. The bone portion need only be large enough to help maintain the three dimensional structure of the collagen portion of the attached cartilage.

The removed joint portion can be any required shape and size, from a small portion of the joint to the entire joint. For example, the joint portion may be circular, rectangular, square, irregularly shaped, or the shape of the entire joint. The joint portion may comprise a focal core, a full or partial intact condyle, a full or partial intact patellar groove, or any other full or partial structure that is found in a joint.

The joint portion could be removed from any joint present in a human cadaver or animal. Specific joints include, but are not limited to, the knee, elbow, ankle, hip, shoulder, wrist, and spine. Any joint which contains a bone and cartilage portion is included in the method of the present invention.

The collagen scaffold structure can be stabilized by exposing the graft to chemical agents called cross-linkers or by glycation. More than one treatment can be used to stabilize the collagen scaffold structure. For example, both cross-linking and glycation can be used; alternatively, or in addition, more than one cross-linker and/or glycating agent may be used. If more than one treatment is used to stabilize the collagen, the treatments may be done either simultaneously or consecutively. If done consecutively, the stabilizing treatments may be done in any order. Specific stabilization treatments may be repeated any number of times.

The graft can be exposed to the stabilizers in any manner, including submerging the entire graft or the cartilage portion thereof in the stabilizer, pouring the stabilizer over the graft or the cartilage portion thereof, placing the graft in a chamber with a vaporized stabilizer, or any other method of contacting the stabilizer with the cartilage.

Cross-linkers that can be used to stabilize the collagen structure include aldehydes, aliphatic and aromatic diamines, carbodiimides and diisocyanates. Specific aldehydes include, but are not limited to, glutaraldehyde, formaldehyde and adipic dialdehyde. In one embodiment, vaporized formaldehyde is used.

The cross-linking agent may be in either liquid or vapor form and exposure to the cross-linking agent may occur in buffered saline or other physiologic buffers at physiologic pH. In one specific embodiment, the graft's collagen matrix is stabilized by fixing the graft with 2% glutaraldehyde in 0.1 M sodium cacodylate and 0.1 M sucrose buffer with pH 7.2 for 48 hours.

The crosslinking treatment can be conducted for varying durations of time. For example, in some embodiments, the cross-linking treatment may last for about several hours to about several days. The cross-linking treatment may be performed at about 4° C., or any other temperature that is suitable for use in a cross-linking treatment. The buffers that may be used in the cross-linking treatment include, but are not limited to, sucrose and sodium cacodylate, water (preferably sterile), phosphate buffered saline, physiologic saline, and diluted alcohol solution. Other buffers may also be suitable. In some embodiments, the buffer can include proteinase inhibitors.

The graft's collagen structure may also be stabilized through the process of glycation by contacting the graft with at least one glycating agent. The level of glycation is determined by the length of time the collagen is in contact with the glycating agent. Any length of time suitable to obtain the desired level of glycation is appropriate for use with the invention. In some embodiments, the glycation treatment may last anywhere from about several hours to about several days. In other embodiments, the glycation process will last for about 3 days to about 7 days. The glycation process may be performed at about 37° C., or any other suitable temperature.

Glycating agents include, but are not limited to, threose, glucose, and ribose. Other reducing sugars are also suitable for use as glycating agents and more than one glycating agent may be used in a single glycation treatment. In addition, other agents, besides sugars, that are suitable for use as glycating agents may also be used to stabilize the collagen structure of the graft.

Physiological buffers suitable for use in the glycation treatment include, but are not limited to, sucrose and sodium cacodylate, water (preferably sterile), phosphate buffered saline, physiologic saline, and diluted alcohol solution. Other various buffers may also be suitable. In some embodiments, the buffer can include proteinase inhibitors.

In certain embodiments, the bone portion of the graft is demineralized. By “demineralized” it is meant that the bone portion of the graft is processed to remove at least a portion of the bone's mineral phase, lipids, blood and/or other cellular materials. The mineral phase can include, but is not limited to, calcium phosphate and/or, more specifically, hydroxyapatite.

The bone portion of the graft can be demineralized by treating it with a demineralizing agent. Demineralizing agents include guanidine hydrochloride, hydrochloric acid, and basic EDTA. More than one demineralizing agent may be used and the treatments may be done either simultaneously or consecutively. For example, a graft may be demineralized by using a treatment of guanidine hydrochloride and then a treatment of hydrochloric acid. Specific demineralization treatments may also be repeated any number of times, if desired. If done consecutively, the demineralization treatments can occur in any order.

The bone portion of the graft may be demineralized in varying amounts, from slightly demineralized to completely demineralized. The level of demineralization is determined by the length of time the bone portion is in contact with a demineralizing agent. In some embodiments the demineralization process may last anywhere from about several minutes to about several hours, depending on the desired amount of demineralization. However, any amount of time suitable for the desired level of demineralization will be appropriate for the present invention. In addition, the graft may, optionally, only be demineralized in a specific area of the bone portion.

The graft may be exposed to the demineralizing agent in any manner. For example, the entire graft may be submerged in the demineralizing agent or the demineralizing agent may be poured over the graft. Any other method of contacting the bone portion with a demineralizing agent is also included in the invention.

One standard method of demineralizing bone involves placing bone segments in 0.6 N hydrochloric acid for 24 hours at a mass per volume ratio of 1 g of bone to 100 mL acid solution and then thoroughly washing the remaining lyophilized bone in sterile water. This standard demineralization technique may be modified by adjusting the acid concentration, adjusting the duration of demineralization, and adjusting the duration of post-demineralization washing.

In addition, a number of solvents may be introduced during the process, such as ethanol, methanol, ether, chloroform and/or different combinations thereof. Solvents may be added during the process to remove fat, bacteria, or any other undesirable material from the graft. Other processes of bone demineralization familiar to those versed in the art and suitable for use with the method of the present invention are also included.

The graft of the present invention may also be devitalized to illicit cellular disruption and remove cellular material. The term “devitalization” refers to any process that causes cellular disruption and/or removes cellular material from a graft. Processes used to devitalize the graft include freezing the graft, treating the graft with alcohol, subjecting the graft to gamma radiation and subjecting the graft to freeze-thaw cycles. Any other method of devitalizing grafts can also be used with the present invention.

The graft may also be devitalized in varying amounts, from slightly devitalized to completely devitalized. If more than one devitalization treatment is applied to devitalize the graft, the treatments may occur in any order or may be done simultaneously. A specific devitalization treatment may also be repeated any number of times. For example, to devitalize the graft, it may first be treated with alcohol, then exposed to gamma radiation, and again treated with alcohol. Specific areas of the graft may also be devitalized, without devitalizing the entire graft.

Accordingly, in one embodiment of the invention the graft may be devitalized by exposing it to gamma radiation in an amount of about 0.5 to 3 MegaRad. The graft may be exposed to gamma radiation for any amount of time.

In another embodiment, the graft may be placed in an alcohol solution. The appropriate alcohols include isopropanal, methanol, ethanol, and any other alcohol suitable for use in a devitalization treatment. For example, the graft may be immersed in isopropanol for about five minutes at room temperature.

In a further embodiment of the invention, the graft may be subjected to freeze-thaw cycles to cause devitalization. For example, the graft of the present invention may be frozen by submersion in liquid nitrogen or by placing it in a freezer. The graft may be thawed by immersion in an isotonic saline bath for about ten minutes at about room temperature or by placing it at room temperature without a saline bath. More than one freeze-thaw cycle may be repeated any number of times.

In addition to the processes described above, other processes for devitalizing cartilage and demineralizing bone known to those skilled in the art are also included in the method of the present invention.

When the graft is both demineralized and devitalized, the demineralization and devitalization treatments may be done in any order, or they can be done simultaneously. When more than one treatment is used to devitalize and/or demineralize the graft, the treatments may be done in any order. For example, the graft may be subjected to one devitalization treatment, then a demineralization treatment, then another devitalization treatment.

Likewise, the demineralization and/or devitalization treatments may occur before, during, or after the collagen stabilization treatment.

In some embodiments, the graft is treated to remove at least a portion of the cellular debris. The removal of cellular debris can be accomplished by treating, and/or submerging the graft in chloroform or other solvents. Only a portion of the cellular debris may be removed up to the entire amount of cellular debris. About 10% to about 100% of the cellular debris is removed, preferably about 20 to about 90%, about 30 to about 80%, about 40 to about 70% or about 50 to about 60% of the cellular debris may be removed.

In some embodiments, at least a portion of the proteoglycans and non-collagenous proteins may be removed, for example, by processing the graft with proteoglycan-depleting enzymes. Only a portion of the proteoglycans and/or non-collagenous proteins may be removed up to the entire amount of proteoglycans and/or non-collagenous. About 10% to about 100% of the proteoglycans and/or non-collagenous are removed, preferably about 20 to about 90%, about 30 to about 80%, about 40 to about 70% or about 50 to about 60% of the proteoglycans and/or non-collagenous are removed.

In some embodiments, removing at least a portion of the proteoglycans and other carbohydrates from the graft alters one or more of the mechanical properties of the cartilage portion of the graft. Preferably, at least a portion of the proteoglycans are removed throughout the collagen portion of the graft, i.e., removing the proteoglycans at the surface and below the surface of the graft. Removing the proteoglycans only at the surface of the graft does not substantially alter the mechanical properties of the graft.

The term “mechanical properties” means the characteristics and/or responses of a substance or structure. To “alter mechanical properties” means to either increase or decrease at least one of the mechanical properties of a substance or structure. For example, one or more of the mechanical properties may be altered by removing at least a portion of the proteoglycans. The mechanical properties that may be substantially altered include, but are not limited to, the compressive properties, tensile properties, swelling properties, shear properties, viscoelastic properties and elastic properties of the cartilage.

In one embodiment, the compressive and/or shear properties of the graft are decreased. In other embodiments, the viscoelastic properties of the graft are decreased. In other embodiments, the graft is less able to recover after deformation.

In some embodiments, one or more mechanical properties of the cartilage portion of the graft are slightly altered. In other embodiments, one or more mechanical properties of the cartilage portion of the graft are substantially altered. One or more mechanical properties of the cartilage portion of the graft may be by about 1% to about 100%, by about 5% to about 95%, by about 10% to about 80%, by about 20% to about 70%, by about 30% to about 60% or by about 40% to about 50%.

Proteoglycan-depleting enzymes include hyaluronidase, aggrecanase, trypsin, chondroitinase, and keratanase. Additionally, the enzymes listed above may be used either alone or in combination with each other. If more than one enzyme is used, they may be used simultaneously or consecutively. If used consecutively, they can be used in any order.

One method used to remove proteoglycans and non-collagenous material from the graft involves dissolving hyaluronidase at concentrations of about 0.1 to about 10 mg/ml in culture medium, buffered saline or trypsin. The graft is then incubated in the enzyme solution at about 37° C. The incubation period can range from about 2 hours to about 48 hours depending on the enzyme concentration. After the incubation period, the graft is rinsed with sterile DI water.

Embodiments of the method of the present invention further include seeding the graft with one or more types of cells. “Seeding” the graft with cells refers to the process of inserting, or placing, one or more types of cells into, or onto, at least a portion of the graft. Preferably, the cells are placed in or on the collagen portion of the graft.

The graft may be seeded at any time with autologous cells, allogeneic cells or a combination of both. More specifically, in certain embodiments, the graft may be seeded with stem cells, mesenchymal cells, progenitor cells, bone marrow cells, synovial cells, osteoblasts, fibroblasts, chondroblasts, chondrocytes, or combinations of these cells. Additionally, other types of cells that are known to those skilled in the art and may have some therapeutic value can also be used to seed the graft.

In certain embodiments, one or more biological agents are added to the graft. By “biological agent” it is meant any agent that has, or produces, biological, physiological and/or pharmaceutical activity upon administration to a living organism. These biological agents may be added to the graft at any time, for example, before or after implantation. Preferably, the biological agents are added after any stabilization, devitalization and/or demineralization treatments.

Suitable biological agents include, but are not limited to, growth factors, cytokines, antibiotics, strontium salts, fluoride salts, calcium salts, sodium salts, bone morphogenetic factors, chemotherapeutic agents, angiogenic factors, osteoconductive agents, chondroconductive agents, inductive agents, painkillers, proteins, peptides, or combinations thereof.

Growth factors that can be added to the graft include platelet derived growth factor (PDGF), transforming growth factor beta (TGFβ), insulin-related growth factor-I (IGF-I), insulin-related growth factor II (IGF-II), beta-2-microglobulin, bone morphogenetic protein (BMP), fibroblast growth factor (FGF), interleukin-1β (IL-1β), hepatocyte growth factor (HGF), cartilage derived morphogenetic protein (CD-MP), growth differentiation factors (GDFs), platelet-rich-plasma (PRP), or combinations of growth factors.

Chondroinductive agents include prostaglandin E2, thyroid hormone, dihydroxy vitamin D, ascorbic acid, dexamethasone, staurosporine, dibutyrl cAMP, concavalin A, vanadate, FK506, or combinations of different chondroinductive agents. Antibiotics include tetracycline hydrochloride, vancomycin, cephalosporins, and aminoglycocides such as tobramycin, gentamicin, and combinations thereof. Pain killers include lidocaine hydrochloride, bipivacaine hydrochloride, ketorolac tromethamine and other non-steroidal anti-inflammatory drugs.

The biological agent added to the graft can also be a protein or combinations of proteins. For example, proteins of demineralized bone, demineralized bone matrix (DBM), bone protein (BP), bone morphogenetic protein (BMP), osteonectin, osteocalcin, osteogenin, or combinations of these proteins can be added to the graft.

Other suitable biological agents include cis-platinum, ifosfamide, methotrexate, doxorubicin hydrochloride, or combinations thereof.

In some embodiments, the graft is further processed. For example, in some embodiments, the graft is sterilized. The graft may be sterilized by exposing the graft to radiation, submerging the graft in alcohol, treating the graft with ethylene oxide, treating the graft with propylene oxide, steam sterilization, and/or any other method for sterilizing grafts known to those skilled in the art. More than one type of sterilization procedure may be performed on the graft and a single sterilization procedure may be repeated more than once. If more than one type of sterilization procedure is performed, then the sterilization procedures may occur in any order.

The graft may be stored by either freezing or lyophilization. When the graft is lyophilized, it may be stored at room temperature. However, any method of storing and preserving grafts known to those skilled in the art is suitable for use in the present invention.

In addition to the embodiments described above, the graft can be modified to either include or exclude the tidemark. The term “tidemark” refers to the line or area that is present between the calcified cartilage and the hyaline cartilage in a joint. The tidemark in the graft may be processed to increase its porosity or may be kept fully intact.

In some embodiments, the bone portion is entirely or partially removed from the graft, preferably, after any collagen stabilization treatments have taken place. Since the collagen structure will already be stabilized, the removal of the bone portion of the graft is not expected to disrupt the collagen structure.

The shape and/or size of the graft can be modified to fit the implant site. This modification can take place at any time. Preferably, this modification takes place after any collagen stabilization treatments. The shape and/or size of the graft can be modified before or after any other treatments, and the modification can even be performed immediately before implantation of the graft.

The present invention also includes grafts made by any of the methods described herein.

The present invention also provides a method of repairing cartilage defects by implanting the graft into a joint with a cartilage defect. The graft of the invention can be implanted into either partial or full-thickness cartilage defects in joints by those of skill in the art. In specific embodiments, the graft is inserted into the joint defect either arthroscopically or through an open incision according to surgical techniques, and using surgical instruments, generally known to those skilled in the art. For example, a surgeon can implant the graft by simply drilling a hole in the defect site, inserting the graft into the hole, and anchoring the graft to the hole. Once the graft is implanted into the defect, it provides a scaffold for the growth of cartilage appropriate cells.

Preferably, the graft is taken from a similar anatomical site as the defect. For example, if the graft is being used to repair a knee defect, then the graft should be taken from a human cadaver or animal knee.

The graft can be inserted into an animal of the same species from which it was derived, or it may be implanted into an animal of a different species. Preferably, the graft is implanted into an animal of the same species from which it was derived.

In one embodiment, the graft is inserted into a cartilage defect so that the split-line orientation of the graft collagen matches the orientation of the cartilage defect. Matching the collagen orientation of the graft and the defect is expected to promote repair at the defect site. Alternatively or in addition, the curvature of the outer surface of the graft may be matched with the curvature of the cartilage defect.

There are also a number of ways in which the graft may be inserted and anchored into the defect site. For example, the graft may be merely press fit into the defect area or an anchor can be used to affix the graft to the defect. In one embodiment, the graft is fixed onto the damaged joint by joining the bone portion of the graft to exposed bone cuts on the joint and using plates, nails, screws, pins and/or adhesives to maintain the graft in place. In other embodiments, the graft is affixed to the defect using pins, sutures, adhesives, organic glues, clotting materials or any other material known to be suitable for affixing cartilage grafts. More than one type of anchor may be used to affix the graft to the cartilage defect site.

Instrumentation of imaging techniques to measure and match the curvature of the articular cartilage may be packaged with the graft as a kit.

The previously described versions of the present invention have many advantages over the prior art. For example, the problem of patient donor site morbidity found in mosaicplasty is overcome by the present invention because the graft is not removed from a patient donor site. Instead, the graft is removed from a cadaver or animal, leaving potential donor sites in the patient unharmed. Removing the graft from cadaver joints also provides a large resource of readily available grafts compared to the limited number of appropriate autograft sites found in patients undergoing mosaicplasty. Lastly, the graft of the invention may be shaped to fit any size defect, including replacement of an entire articulating joint.

In versions of the invention where the graft is devitalized, the risk of patient immune response to the implanted graft is either greatly decreased or completely removed. Furthermore, the process of devitalization reduces the risk that diseases will be transferred to the patient through implantation of the graft.

Once the graft is implanted into a cartilage defect site, the bone portion of the graft promotes adhesion to the defect site. Preservation of the graft's three dimensional collagen structure provides additional advantages over the prior art. Since the natural collagen structure of the graft is substantially maintained, the graft is expected to promote the growth of articular cartilage having similar mechanical properties to native articular cartilage. Thus, the graft is expected to return normal cartilage function to the defect site.

Preservation of the three dimensional collagen structure also allows a surgeon to match the collagen orientation of the graft with the orientation of the cartilage at the defect site. Matching the collagen orientation of the graft and defect site should increase the quality and duration of cartilage repair because the graft is oriented correctly compared to the surrounding native collagen. Matching the collagen orientation may also improve integration with the surrounding tissue as the correctly oriented graft may be better recognized by the surrounding cartilage. Thus, unlike the prior art, the graft of the present invention can potentially restore normal joint function for extended periods of time in individuals suffering from articular cartilage defects. The advantages outlined above are not required in every embodiment of the invention and are only present to illustrate the potential advantages of the present invention.

The following examples further illustrate the invention and should not be construed as limiting.

### EXAMPLE 1

In this example a graft with a three dimensional collagen structure substantially similar to native articular collagen is produced.

A graft composed of bone and cartilage is resected from the knee joint. The graft is washed in physiologic saline to remove unwanted proteins and other water soluble materials.

The collagen matrix of the graft is stabilized by exposing the graft to 2.5% glutaraldehyde in 0.1 M sodium cacodylate buffer at pH 7.2 for 44-48 hours. The graft is then washed sequentially in large volumes of buffered saline solution for 2 hours.

To remove proteoglycans and other non-collagenous proteins, the graft is incubated in hyaluronidase (0.2-0.5 mg/mL) and trypsin (0.25%) for 18-20 hours at 37° C. while gently agitated. Afterward, the graft is washed several times in physiological saline.

The bone portion of the graft is demineralized by placing the bone in 0.6 N hydrochloric acid for 1 hour at a volume per volume ratio of 35:1 to 100:1. The acid is then removed by washing the graft in physiologic saline.

Following demineralization, the graft is dehydrated using ethanol. Finally, the graft is frozen in liquid nitrogen or lyophilized for storage.

### EXAMPLE 2

In this example a graft with a three dimensional collagen structure substantially similar to native human collagen is produced.

A graft composed of bone and cartilage is resected from the knee joint of a human cadaver under aseptic conditions. The graft is washed in sterile water to remove unwanted proteins and other water soluble materials. The graft is submerged in isopropanol for at least five minutes to cause cellular disruption and death. The graft is also submerged in chloroform to remove cellular debris.

To remove proteoglycans and other non-collagenous proteins, the graft is incubated in hyaluronidase (2-5 mg/mL) and trypsin (0.1-0.25%) for 2-18 hours at 37° C. Afterward, the graft is washed several times in physiological saline.

The bone portion of the graft is demineralized by placing the bone in 0.6 N hydrochloric acid for 24 hours at a mass per volume ratio of 1 g of bone to 100 mL acid solution. The acid is then removed by washing the graft in sterile water.

Following demineralization, the collagen matrix of the graft is stabilized by exposing the graft to 2% glutaraldehyde in 0.1 M sodium cacodylate and 0.1 M sucrose buffer with pH 7.2 for 48 hours. The graft is then washed in buffered saline solution for 12 hours and processed using proteoglycan depleting agents for 18 more hours at 37° C. Next, the graft is washed in buffered saline again and dehydrated using ethanol. Finally, the graft is frozen in liquid nitrogen for storage.

### EXAMPLE 3

In this example an alternate method of making graft with a three dimensional collagen structure substantially similar to native human collagen is presented.

A graft composed of bone and cartilage is resected from the knee joint of a human cadaver under aseptic conditions. The graft is washed in sterile water to remove unwanted proteins and other water soluble materials. After washing the graft in sterile water, it is submerged in isopropanol for at least five minutes to cause cellular disruption and death. It is then washed in sterile water and exposed to gamma radiation in an amount of about 0.5 to 3 MegaRad for a predetermined quantity of time. The graft is submerged in isopropanol again and washed with sterile water.

To remove proteoglycans and other non-collagenous proteins, the graft is incubated in hyaluronidase (2-5 mg/mL) and trypsin (0.1-0.25%) for 2-18 hours at 37° C. Afterward, the graft is washed several times in physiological saline.

The bone portion of the graft is demineralized by immersing the bone in 0.5 M disodium EDTA at 4° C., which adjusted to pH 8.3 with 10 M NaOH. The EDTA solution is replaced every 24 hours.

Following demineralization, the collagen matrix of the graft is stabilized through exposure to cross-linkers. Specifically, the collagen matrix is stabilized by exposing the graft to 2% glutaraldehyde in 0.1 M sodium cacodylate and 0.1 M sucrose buffer with pH 7.2 for 48 hours. The graft is then washed in buffered saline solution for 12 hours and processed using proteoglycan depleting agents for 18 more hours at 37° C. Next, the graft is washed in physiological saline again and frozen and lyophilized for storage.

### EXAMPLE 4

In this example, an alternate method of making graft with a three dimensional collagen structure substantially similar to native human collagen is presented.

A graft composed of bone and cartilage is resected from the knee joint of a human cadaver under aseptic conditions. The graft is washed in sterile water to remove unwanted proteins and other water soluble materials. After washing the graft in sterile water, it is submerged in isopropanol for at least five minutes to cause cellular disruption and death. It is then washed in sterile water and exposed to gamma radiation in an amount of about 0.5 to 3 MegaRad for a predetermined quantity of time. The graft is submerged in isopropanol again and washed with sterile water.

To remove proteoglycans and other non-collagenous proteins, the graft is incubated in hyaluronidase (2-5 mg/mL) and trypsin (0.1-0.25%) for 2-18 hours at 37° C. Afterward, the graft is washed several times in physiological saline.

Following the physiological saline wash, the collagen matrix of the graft is stabilized through exposure to cross-linkers. Specifically, the collagen matrix is stabilized by exposing the graft to 2% glutaraldehyde in 0.1 M sodium cacodylate and 0.1 M sucrose buffer with pH 7.2 for 48 hours. The graft is then washed in buffered saline solution for 12 hours and processed using proteoglycan depleting agents for 18 more hours at 37° C. The graft is not exposed to any demineralization treatment. Next, the graft is washed in physiological saline again and frozen and lyophilized for storage.

### EXAMPLE 5

In this example, the proteoglycan content of a caprine knee tissue graft was reduced.

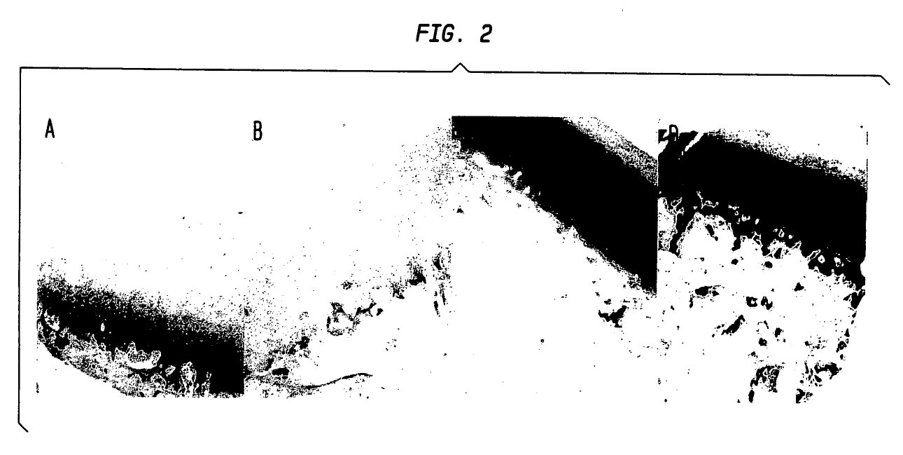

Grafts were digested in a hyaluronidase/trypsin solution (0.35 mg/mL hyaluronidase in a 0.25% trypsin/EDTA solution) or in hyaluronidase alone (0.5 mg/mL hyaluronidase in Phosphate Buffered Saline) for approximately 20 hours at 37° C. The grafts were then histologically stained with Safranin-O. Results are shown in FIG. 2.

As shown, samples digested in the hyaluronidase/trypsin solution were practically devoid of GAG (Samples A and B). Proteoglycan content decreased only superficially in samples digested with hyaluronidase alone.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

The entire disclosures of publications mentioned herein are hereby incorporated by reference herein in their respective entireties.

1. An articular cartilage graft comprising a collagen portion, wherein said collagen has substantially the same three-dimensional collagen architecture as native human articular cartilage.

2. A method for making the graft of claim 1, comprising:
removing at least a portion of a joint from a human cadaver to form a graft, said graft comprising a cartilage portion and a bone portion; and
treating said graft to stabilize the collagen scaffold structure.

3. The method of claim 2, wherein said portion of a joint comprises a focal core.

4. The method of claim 2, wherein said portion of a joint comprises a structure selected from the group consisting of an intact condyle or portion thereof, and an intact patellar groove or portion thereof.

5. The method of claim 2, wherein said portion of a joint is an entire joint.

6. The method of claim 2, wherein said cartilage portion of said graft and said bone portion of said graft are separated by a tidemark.

7. The method of claim 2, wherein said joint is selected from the group consisting of:
a) knee;
b) elbow;
c) ankle;
d) hip;
e) shoulder;
f) wrist;
g) finger; and
h) spine.

8. The method of claim 2, wherein said treating comprises using a crosslinker selected from the group consisting of an aldehyde; glycation; an aromatic diamine; and a diisocyanate.

9. The method of claim 8, wherein said glycation is performed using a substance selected from the group consisting of ribose, threose, and other sugars.

10. The method of claim 2, further comprising devitalizing said graft.

11. The method of claim 10, wherein said graft is devitalized by a process selected from the group consisting of: freezing the graft, treating the graft with alcohol, subjecting the graft to gamma radiation, and subjecting the graft to freeze-thaw cycles.

12. The method of claim 11, further comprising treating said graft to remove at least a portion of the cellular debris.

13. The method of claim 12, wherein said cellular debris is removed by submerging the graft in chloroform.

14. The method of claim 2, further comprising demineralizing the bone portion of the graft.

15. The method of claim 14, wherein said demineralization comprises treating the graft with a material selected from the group consisting of: guanidine hydrochloride, hydrochloric acid, and basic EDTA.

16. The method of claim 2, further comprising treating said graft to remove at least a portion of the proteoglycans and non-collagenous proteins.

17. The method of claim 16, wherein said treating to remove proteoglycans and non-collagenous proteins comprises processing the graft with an enzyme selected from the group consisting of hyaluronidase, aggrecanase, trypsin, chondroitinase, keratanase, and combinations thereof.

18. The method of claim 2, further comprising seeding the graft with cells.

19. The method of claim 18, wherein said cells are selected from the group consisting of stem cells, mesenchymal cells, progenitor cells, bone marrow cells, synovial cells, osteoblasts, fibroblasts, chondroblasts and chondrocytes.

20. The method of claim 2, further comprising adding one or more biological agents to said graft.

21. The method of claim 20, where said biological agents are selected from the group consisting of: a growth factor, a cytokine, an antibiotic, a strontium salt, a fluoride salt, a calcium salt, a sodium salt, a bone morphogenetic factor, a chemotherapeutic agent, an angiogenic factor, an osteoconductive agent, a chondroconductive agent, a painkiller, and combinations thereof.

22. The method of claim 21, wherein said growth factor is selected from the group consisting of: platelet derived growth factor (PDGF), transforming growth factor beta (TGFb), insulin-related growth factor-I (IGF-I), insulin-related growth factor II (IGF-II), beta-2-microglobulin (BDGF-II), bone morphogenetic protein (BMP), fibroblast growth factor (FGF), interleukin-1beta (IL-1b), hepatocyte growth factor (HGF), cartilage derived morphogenetic protein (CD-MP), growth differentiation factors (GDFs), platelet-rich-plasma (PRP), or a combination thereof.

23. The method of claim 21, wherein said chondroconductive agent is selected from the group consisting of prostaglandin E2, thyroid hormone, dihydroxy vitamin D, ascorbic acid, dexamethasone, staurosporine, dibutyrl cAMP, concavalin A, vanadate, FK506, and combinations thereof.

24. The method of claim 21, wherein said antibiotic is selected from the group consisting of tetracycline hydrochloride, vancomycin, cephalosporins, and aminoglycocides such as tobramycin, gentamicin, and combinations thereof.

25. The method of claim 20, wherein said biological agent is selected from the group consisting of proteins of demineralized bone, demineralized bone matrix (DBM), bone protein (BP), bone morphogenetic protein (BMP), osteonectin, osteocalcin, osteogenin, and combinations thereof.

26. The method of claim 20, wherein said biological agent is selected from the group consisting of cis-platinum, ifosfamide, methotrexate, doxorubicin hydrochloride, and combinations thereof.

27. The method of claim 21, wherein said pain killer is selected from the group consisting of lidocaine hydrochloride, bipivacaine hydrochloride, non-steroidal anti-inflammatory drugs, and combinations thereof.

28. The method of claim 27, wherein said non-steroidal anti-inflammatory drug is ketorolac tromethamine.

29. The method of claim 2, further comprising removing the attached bone portion of the graft to create a cartilage-only scaffold.

30. A graft prepared by the method of claim 2.

31. A method of repairing a cartilage defect, comprising:
inserting the graft of claim 1 into a partial or full-thickness cartilage defect in a joint.

32. The method of claim 31, wherein the orientation of the collagen in said graft matches the split-line orientation of the cartilage defect of the joint.

33. The method of claim 31, wherein the curvature of the outer surface of said graft matches the curvature of the cartilage defect of the joint.

34. The method of claim 31, wherein said graft is press-fit into the defect.

35. The method of claim 31, further comprising using an anchor to affix said graft in said defect.

36. The method of claim 35, wherein said anchor is selected from the group consisting of a pin, sutures, anchor, adhesives, organic glue, and clotting material.

37. The method of claim 31, wherein said graft is implanted arthroscopically.

38. The method of claim 31, wherein said graft is implanted through an open incision.

39. The method of claim 31, wherein said more than one graft is inserted into a partial or full-thickness cartilage defect in a joint.

40. A method for making an articular cartilage graft, comprising:
removing at least a portion of a joint from an animal to form a graft, said graft comprising a cartilage portion and a bone portion;
treating said graft to stabilize the collagen scaffold structure; and
removing at least a portion of the proteoglycans from said graft, wherein at least one of the mechanical properties of said cartilage portion of said graft is substantially different than at least one of the mechanical properties of native cartilage.

41. The method of claim 40, where said mechanical properties are selected from the group consisting of: compressive properties, tensile properties, swelling properties, shear properties, and elastic properties.

42. The method of claim 40, where said proteoglycans are removed from said graft by treating said graft with an enzyme selected from the group consisting of hyaluronidase, aggrecanase, trypsin, chondroitinase and keratanase.

43. The method of claim 40, wherein at least about 50% of the proteoglycans are removed from said graft.

44. The method of claim 40, wherein at least 90% of the proteoglycans are removed from said graft.

45. The method of claim 40, wherein said portion of a joint comprises a focal core.

46. The method of claim 40, wherein said portion of a joint comprises a structure selected from the group consisting of an intact condyle or portion thereof, and an intact patellar groove or portion thereof.

47. The method of claim 40, wherein said portion of a joint is an entire joint.

48. The method of claim 40, wherein said cartilage portion of said graft and said bone portion of said graft are separated by a tidemark.

49. The method of claim 40, wherein said joint is selected from the group consisting of:
a) knee;
b) elbow;
c) ankle;
d) hip;
e) shoulder;
f) wrist;
g) finger; and
h) spine.

50. The method of claim 40, wherein said treating comprises using a crosslinker selected from the group consisting of an aldehyde; glycation; an aromatic diamine; and a diisocyanate.

51. The method of claim 50, wherein said glycation is performed using a substance selected from the group consisting of ribose, threose, and other sugars.

52. The method of claim 40, further comprising devitalizing said graft.

53. The method of claim 52, wherein said graft is devitalized by a process selected from the group consisting of: freezing the graft, treating the graft with alcohol, subjecting the graft to gamma radiation, and subjecting the graft to freeze-thaw cycles.

54. The method of claim 53, further comprising treating said graft to remove at least a portion of the cellular debris.

55. The method of claim 54, wherein said cellular debris is removed by submerging the graft in chloroform.

56. The method of claim 40, further comprising demineralizing the bone portion of the graft.

57. The method of claim 56, wherein said demineralization comprises treating the graft with a material selected from the group consisting of: guanidine hydrochloride, hydrochloric acid, and basic EDTA.

58. The method of claim 40, further comprising seeding the graft with cells.

59. The method of claim 58, wherein said cells are selected from the group consisting of stem cells, mesenchymal cells, progenitor cells, bone marrow cells, synovial cells, osteoblasts, fibroblasts, chondroblasts and chondrocytes.

60. The method of claim 40, further comprising adding one or more biological agents to said graft.

61. The method of claim 60, where said biological agents are selected from the group consisting of: a growth factor, a cytokine, an antibiotic, a strontium salt, a fluoride salt, a calcium salt, a sodium salt, a bone morphogenetic factor, a chemotherapeutic agent, an angiogenic factor, an osteoconductive agent, a chondroconductive agent, a painkiller, and combinations thereof.

62. The method of claim 61, wherein said growth factor is selected from the group consisting of: platelet derived growth factor (PDGF), transforming growth factor beta (TGFb), insulin-related growth factor-I (IGF-I), insulin-related growth factor II (IGF-II), beta-2-microglobulin (BDGF-II), bone morphogenetic protein (BMP), fibroblast growth factor (FGF), interleukin-1beta (IL-1b), hepatocyte growth factor (HGF), cartilage derived morphogenetic protein (CD-MP), growth differentiation factors (GDFs), platelet-rich-plasma (PRP), or a combination thereof.

63. The method of claim 61, wherein said chondroconductive agent is selected from the group consisting of prostaglandin E2, thyroid hormone, dihydroxy vitamin D, ascorbic acid, dexamethasone, staurosporine, dibutyrl cAMP, concavalin A, vanadate, FK506, and combinations thereof.

64. The method of claim 61, wherein said antibiotic is selected from the group consisting of tetracycline hydrochloride, vancomycin, cephalosporins, and aminoglycocides such as tobramycin, gentamicin, and combinations thereof.

65. The method of claim 61, wherein said biological agent is selected from the group consisting of proteins of demineralized bone, demineralized bone matrix (DBM), bone protein (BP), bone morphogenetic protein (BMP), osteonectin, osteocalcin, osteogenin, and combinations thereof.

66. The method of claim 61, wherein said biological agent is selected from the group consisting of cis-platinum, ifosfamide, methotrexate, doxorubicin hydrochloride, and combinations thereof.

67. The method of claim 61, wherein said pain killer is selected from the group consisting of lidocaine hydrochloride, bipivacaine hydrochloride, non-steroidal anti-inflammatory drugs, and combinations thereof.

68. The method of claim 67, wherein said non-steroidal anti-inflammatory drug is ketorolac tromethamine.

69. A graft prepared by the method of claim 40.
